(19) United States
(12) Patent Application Publication
(10) Pub. No.: US 2024/0033672 A1
(43) Pub. Date: Feb. 1, 2024

(54) FILTER TEST DEVICE FOR A PORTABLE AIR PURIFIER

(71) Applicant: Dyson Technology Limited, Wilshire (GB)

(72) Inventor: Nikian Naji AGHABABAIE, London (GB)

(73) Assignee: Dyson Technology Limited, Wiltshire (GB)

(21) Appl. No.: 18/267,723

(22) PCT Filed: Oct. 7, 2021
(86) PCT No.: PCT/GB2021/052595

§ 371 Date: Jun. 15, 2023

(30) Foreign Application Priority Data
Dec. 18, 2020 (GB) 2020102.6

(51) Int. Cl. B01D 46/00; A61L 9/20; B01D 46/44; B01D 46/42

(52) U.S. Cl. CPC B01D 46/0086; A61L 9/20; B01D 46/446; B01D 46/442; B01D 46/4245; A61L 2209/14; A61L 2209/111; A61L 2209/12; B01D 2273/18

(57) ABSTRACT

A test device is provided for an air filter comprised in an air channel of a portable air purifier. The test device includes a sensor inlet and a sensor outlet, at least the sensor inlet being connectable to the air channel of the portable air purifier. The test device further includes a filter condition sensor, positioned between the sensor inlet and the sensor outlet, and configured to analyse a condition of an air filter comprised in the air channel.

### TECHNICAL FIELD

The present invention relates to a test device for a filter of a portable air purifier and to a storage case for a portable air purifier comprising such a test device.

### BACKGROUND

Over recent years, public interest in air quality in both indoor and outdoor environments has increased. As a result, many people have been buying air purifying devices for their homes and offices. Air purifiers generally use a compressor to take in air from the direct environment of the air purifier, one or more filters to remove unwelcome particles and other contaminants, and one or more nozzles from which to expel the filtered outgoing air. Depending on the application, the air may not just be filtered, but also cooled, heated, moisturised, dried, or otherwise treated, while it flows between the air inlet and an outlet of the air purifier.

In the international patent application published as WO 2020/021231, a head wearable air purifier is disclosed. The head wearable air purifier, which also comprises speaker assemblies to double as a pair of headphones, comprises an impeller for drawing air in through an air inlet and a filter assembly and for expelling the air from an air outlet downstream from the filter assembly. An arcuate nozzle is connected to the air outlet to guide the expelled air towards an array of nozzle outlets that, in use, are arranged in front of a user's mouth to ensure that the user breathes in clean and filtered air.

Over time, the filters of such air purifiers slowly increase their air resistance due to contaminants in the air being collected on the surface of the filter media. Depending on the type of filter used, in order to keep the restriction low, the filters need to either be washed or replaced.

It is important to keep the air restriction across the filter low to ensure that the compressor can deliver adequate clean air to the user. If the restriction is too high, the compressor could be operating at maximum capacity and the user may not receive the clean air that they require to keep them safe and healthy. As a result it is very important to know when the filter needs to be maintained. Conversely, cleaning or replacing filters too often can lead to a waste of energy, damage to the filter media and may be expensive for the user. To improve the timing of the filter cleaning and replacement, the air purifier may comprise sensor systems for monitoring the filter status. Possible technical solutions to assess the filter status may, e.g., comprise monitoring a pressure difference between different locations in the air flow path, or using sensors for measuring particular particles or gases in the air downstream the filter assembly.

While such technical solutions may be very useful in a desktop or floor model air purifier, they can be challenging to integrate into a more portable or wearable device that is designed to be compact and lightweight. Furthermore, the operation of such filter status monitoring technology may increase the electric power consumption of the usually battery powered portable device.

It is an aim of the present invention to address one or more disadvantages associated with the prior art.

### SUMMARY OF THE INVENTION

According to an aspect of the invention there is provided a test device for an air filter in an air channel of a portable air purifier. The test device comprises a sensor inlet and a sensor outlet, at least the sensor inlet being connectable to the air channel of the portable air purifier. The test device further comprises a filter condition sensor, positioned between the sensor inlet and the sensor outlet, and configured to analyse a condition of the air filter.

By providing a separate test device to which the portable air purifier can be connected for testing the air purifier filters, the portable air purifier can be kept compact and lightweight without losing the possibility to regularly check the condition of its filters. This ensures that the filters can be cleaned or replaced exactly when needed. Because, with this invention, compactness is much less a design constraint, it is further possible to use higher quality sensors for lower costs than would be possible if the filter condition sensor needs to be integrated in the portable air purifier. Also, because the test device is separate from the portable air purifier, it is possible to use it with multiple different air purifiers and not every air purifier needs its own filter condition sensor which can otherwise be costly.

In the context of the current invention, the term ‘portable’ is to be interpreted as being sufficiently compact and lightweight to be carried around by its user while walking. Because of its portability, the air purifier is preferably cordless and battery powered, but corded air purifiers may be portable too. Although the invention is developed for and primarily beneficial when used in combination with a portable air purifier, its use may not be restricted to portable air purifiers only. For example, desktop model and floor model air purifiers may be connected to the same test device. In a special embodiment, the portable air purifier may be wearable, e.g. on the user's head, integrated in a hat, a helmet or a pair of headphones. Wearable air purifiers can be used while keeping the user's hands free for other tasks.

In an embodiment of the invention, the test device comprises an internal pneumatic track connecting the sensor inlet to the sensor outlet, the filter condition sensor being positioned along the internal pneumatic track. The test device further comprises a first pneumatic track, connected to the sensor inlet and configured to be coupled to an air outlet of the portable air purifier. The test device may further comprise a second pneumatic track, connected to the sensor outlet and configured to be coupled to an air inlet of the portable air purifier. This test device can be readily connected to the air inlet and air outlet of a compatible portable air purifier. Adapters may be provided for easy connection to other portable (and non-portable) air purifiers.

When connected, the air channel of the portable air purifier and the first, second, and internal pneumatic track together form a closed loop. The filter to be tested and the filter condition sensor are both part of this closed loop. The air flowing through the filter and the air channel of the air purifier reaches the sensor inlet through the first pneumatic track. In the test device, the air then flows through the internal pneumatic track, where the filter condition sensor obtains measurement data representative of the current condition of the air purifier sensor. After leaving the test device, the air then returns to the air purifier through the second pneumatic track. When only the sensor inlet is connected to the air channel of the portable air purifier, the sensor outlet is in direct communication with the open air.

The filter condition sensor may, e.g., comprise a pressure sensor, a particulate matter sensor, or a gas sensor. When the filter in the air purifier gets saturated, this will hamper the free flow of air through the air channel. As a result, a pressure difference between the upstream and the downstream side of the filter will occur. In a closed loop system, wherein the sensor outlet is connected to the air inlet of the portable air purifier, this pressure difference can be measured by one or more pressure sensors in the test device. Alternatively, in a non-closed loop system, the pressure difference between ambient and the test device pressure sensor can be measured. When the filter is used, this pressure difference will change over time. While ambient pressure may vary over time too, this variation will typically be within an acceptable tolerance range and thus still allow the filter condition to be monitored with a pressure sensor. Software algorithms, possibly using ambient pressure measurements and/or real-time weather data as input, may be used for adjusting for the variation in ambient pressure.

The filter is provided for extracting and trapping particulate matter from the air flowing through the air channel portable air purifier. When used for a longer period, the filter gradually loses its capacity to trap more particulate matter due to the air restriction being too high. When that happens, some of the particulate matter previously trapped in the filter may detach therefrom. A particulate matter sensor can monitor a particulate matter concentration in the air flowing through the test device. The sensor data obtained from the particulate matter sensor can provide an indication of the current condition of the filter in the air channel of the air purifier when measured over time. Gas sensors may, e.g., be useful to detect volatile organic compounds (VOCs) that are released from a partly or fully saturated carbon filter. Combinations of two or more different sensing technologies may lead to more accurate and reliable results.

The test device may further comprise a power source, coupled to the filter condition sensor for providing electrical power thereto. The power source may, e.g., be a battery, comprised in the housing of the test device, or it may be a power connector, configured for connection to an external power source by wire or via wireless inductive coupling. In addition to the filter condition sensor, the power source may, e.g., power a controller or a small display screen. A power connector may be connected to the power source and configured to be connected to a power input of the portable air purifier. The test device, which may not have to be as compact and lightweight as the portable air purifier may, e.g., be used for charging a battery of the portable air purifier or to power a compressor of the air purifier when testing the filter condition. Alternatively, the test device may have its own compressor and for use in the test procedure.

In a special embodiment, the test device comprises at least one docking bay. The docking bay is connected to the second pneumatic track and shaped for holding at least a part of the portable air purifier in a predetermined position, such that the air inlet of the portable air purifier is fully covered by the docking bay. When the inlet of the portable air purifier is fully covered by the docking bay, the outlet can be connected to the first pneumatic track to form the closed loop that is used for testing the filter condition. The connection to the outlet of the portable air purifier may, e.g., be realised through a separate port in the same docking bay, or through a flexible tube that can be connected to the outlet.

According to another aspect of the invention, a combination of a portable air purifier and a test device as described above is provided. With the air outlet of the portable air purifier being coupled to the sensor inlet and the air inlet of the portable air purifier being coupled to the sensor outlet, the filter condition can be tested. In this configuration, a purifier controller of the portable air purifier may be operatively coupled to the filter condition sensor of the test device to control a compressor that produces an airflow through the air channel and the test device. The compressor used for this test may be the compressor that is already provided in the air purifier for its normal air purifying operation. Alternatively, the test device comprises a compressor for providing the air flow needed for the filter condition test.

According to a further aspect of the invention a storage case for a portable air purifier may be provided, comprising a test device according as described above. The storage case may be a portable case for holding and protecting the air purifier while travelling. Alternatively, it may be a part of a docking and charging station that is kept at home. Preferably, the storage case has some additional storage space for holding useful accessories. The storage case may comprise a power source, coupled to the test device for providing electrical power thereto. This power source may be a rechargeable battery and/or a power connector for connecting to a power socket or external power source. If the storage case comprises a rechargeable battery, this may be used for charging the portable air purifier too. In addition to its protective and filter testing functionality, it may thus help to increase the amount of time the air purifier can be used without having to connect it to a wall socket.

In a special embodiment, the storage case further comprises at least one light source for emitting light in a violet portion of the visual spectrum, the at least one light source being arranged in such a way as to illuminate at least a portion of the portable air purifier. The emitted light preferably has a wavelength of about 405 nm, which is very suitable for decontaminating, e.g., the nozzles of the air purifier. An advantage of using light in this part of the visual spectrum for decontaminating parts of the portable air purifier is that it is safer for human interaction than, e.g., ultraviolet light. Light guides inside the storage case may be configured to guide the light emitted by the at least one light source to those portions of the portable air purifier that are either likely to be contaminated or to pass on any contamination to the user.

### BRIEF DESCRIPTION OF THE DRAWINGS

Embodiments of the invention will now be described by way of example with reference to the accompanying drawings, in which:

FIG. 1 shows a schematic representation of a test device and a portable air purifier according to an embodiment of the invention.

FIG. 2 shows a perspective view of an opened storage case for a portable air purifier.

FIG. 3 shows the storage case of FIG. 2, with the portable air purifier in its stored position.

FIG. 4 shows a top view of the filled storage case of FIG. 3.

### DETAILED DESCRIPTION

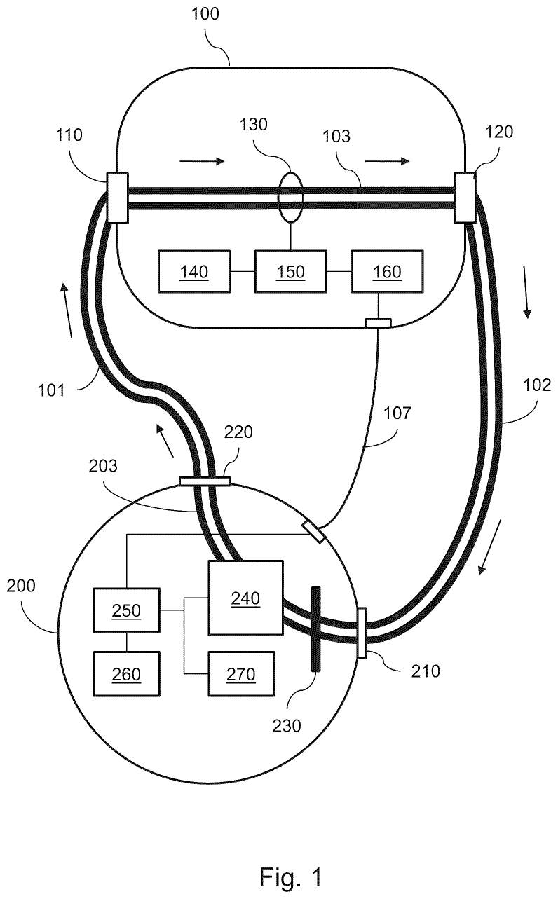

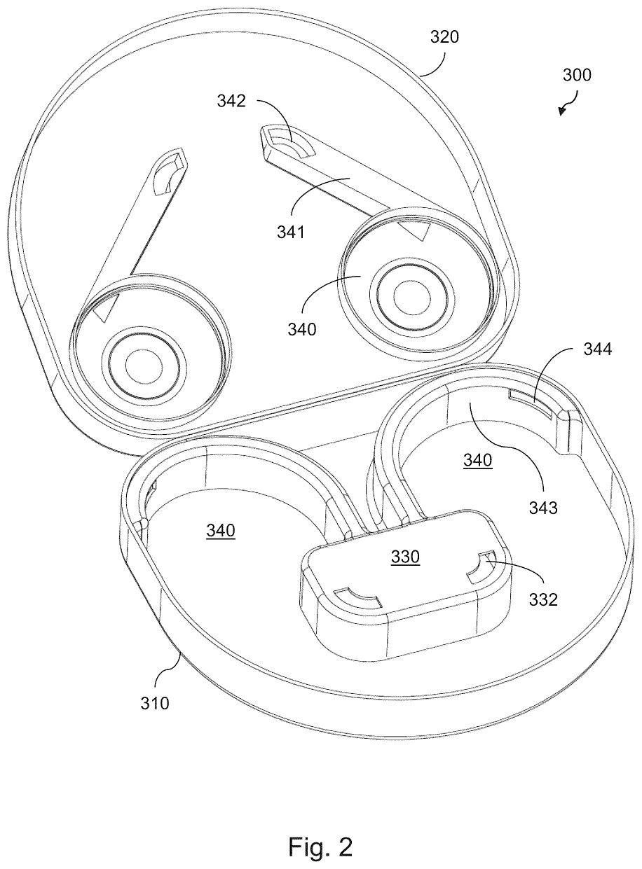

FIG. 1 shows a schematic representation of a test device 100 and a portable air purifier 200 according to an embodiment of the invention. The test device 100 of FIG. 1 comprises a sensor inlet 110 and a sensor outlet 120, both being connectable to an air channel 203 of the portable air purifier 200 via pneumatic lines 101, 102. The pneumatic lines 101, 102 may, e.g., be realised as flexible tubes with connectors at their outer ends for connection to complementary connectors at the inlet 210 and outlet 220 of the air purifier 200. Adapters may be provided for easy connection to portable (and non-portable) air purifiers with differently designed inlets and outlets. In other embodiments, for example as will be discusses below with reference to FIGS. 2 to 4, the pneumatic lines 101, 102, may be rigid channels provided in a storage case 300 or docking station adapted for holding the portable air purifier 200. Inside the test device 100, an internal pneumatic channel 103 connects the sensor inlet 110 to the sensor outlet 120. The test device 100 further comprises a filter condition sensor 130, positioned in or along the internal pneumatic channel 103 and is configured to analyse a condition of an air filter 230 comprised in the air channel 203 of the portable air purifier 200. Together, the two pneumatic lines 101, 102, the internal pneumatic channel 103 and the air channel 203 form a closed loop circuit through which air will flow in the direction of the arrows shown in FIG. 1, when the filter 230 is being tested. The closed loop is preferably completely airtight to avoid any external influences on the pressure or the composition of the air inside the loop. However, depending on the sensor technology used and the required accuracy of the measurements performed, a limited amount of leakage may be acceptable.

The filter condition sensor 130 may, e.g., be a pressure sensor, a particulate matter sensor, or a gas sensor. When the filter 230 in the air purifier 200 gets saturated, this will hamper the free flow of air through the air channel 203. As a result, a pressure difference between the upstream and the downstream side of the filter 230 will occur. This pressure difference can be measured by one or more pressure sensors in the test device 100. The filter 230 is provided for extracting and trapping particulate matter from the air flowing through the air channel 203 of the portable air purifier 200. When used for a longer period of time, the filter 203 gradually loses its capacity to trap more particulate matter and some of the particulate matter previously trapped in the filter may detach therefrom. A particulate matter sensor can monitor a particulate matter concentration in the air flowing through the test device 100. The sensor data obtained from a particulate matter sensor thus provides a clear indication of the current condition of the filter 230 in the air channel 203 of the air purifier 200. Gas sensors may, e.g., be useful to detect volatile organic compounds (VOCs) that are released from a partly or fully saturated carbon filter. Combinations of two or more different sensing technologies may lead to more accurate and reliable results.

In an alternative embodiment, the pneumatic line 102 connecting the sensor outlet 120 to the test purifier inlet 210 may be dispensed with. In such an open loop configuration, the air that has been drawn through the air channel 203 of the air purifier and the internal pneumatic channel of the test device 100 for testing the filter 230 is expelled into the ambient air when leaving the sensor outlet 120. In such an embodiment, the pressure difference between ambient and the test device pressure sensor 130 will change over time when the filter 130 is used. This can be measured with the pressure sensor 130 and used to determine the gradual decline in the filter condition. While ambient pressure may vary over time too, this variation will typically be within an acceptable tolerance range and thus still allow the filter condition to be monitored with a pressure sensor. Software algorithms, possibly using ambient pressure measurements and/or real-time weather data as input, may be used for adjusting for the variation in ambient pressure. Also particulate measure sensors and gas sensors can be used in an open loop configuration.

The test device 100 may further comprise a power source 160, coupled to the filter condition sensor 130 for providing electrical power thereto. The power source 160 may, e.g., be a battery 160, comprised in the housing of the test device 100, or it may be a power connector, configured for connection to an external power source. In addition to the filter condition sensor 130, the power source 160 may, e.g., power a device controller 150 for controlling the filter testing or a small display screen 140 for providing information about the testing. The display screen 140 may be part of a user interface that allows the user to control the functionality of the test device 100. Alternatively, a user interface and/or an electronic controller 250 of the air purifier 200 may control the functioning of the test device 100. In further embodiments, the control functions may be shared between the device controller 150 of the test device and the electronic controller 250 of the air purifier 200. User control may be made possible through both user interfaces. Communication between the test device 100 and the air purifier 200 may take place over a connection line 107 or wireless, via, e.g., Bluetooth.

The connection line 107 may further be used for charging a battery 260 of the air purifier 200. When the test device 100 can be used for charging the air purifier 200, this may allow for longer periods of use of the air purifier 200 without having to plug it into a wall socket. Additionally, it may make it possible to use the compressor 240 of the air purifier 200 for testing the filter condition, even when the battery 260 of the air purifier 200 is empty. The latter may also be made possible by adding a compressor to the test device 100, such that the compressor 240 of the air purifier 200 is not needed for performing a filter condition test.

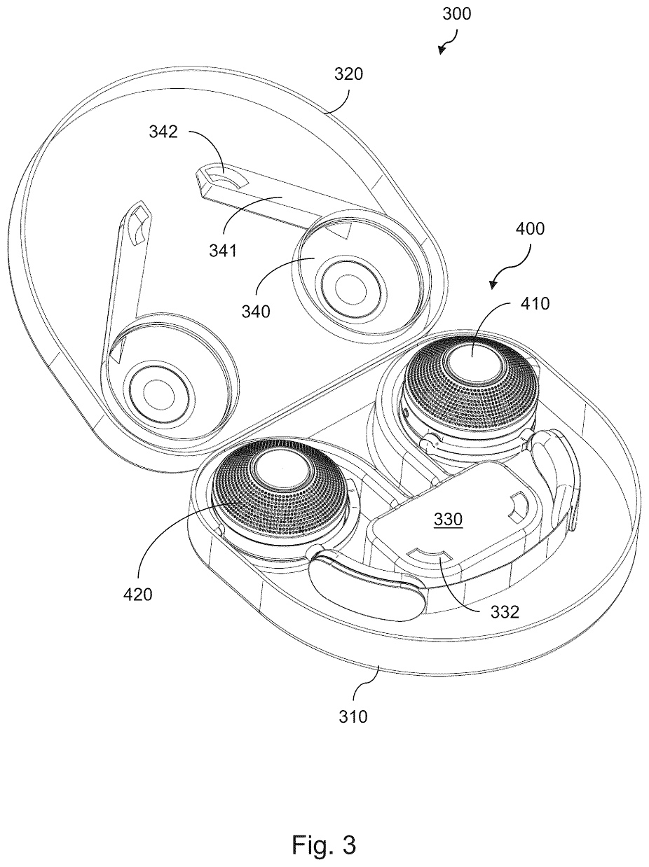

FIGS. 2 and 3 show a perspective view of an opened storage case 300 for a portable air purifier 400. FIG. 2 shows the storage case 300 when empty. FIG. 3 shows the storage case with the air purifier 400 in its storage position. The storage case 300 comprises a bottom part 310 and a cover 320 that are both designed to match the shape of this particular type of portable air purifier 400. The portable storage case 300 comprises a test device 330, similar to the one described above with reference to FIG. 1. While this portable storage case 300 is designed for holding and protecting the air purifier 400 while travelling, alternative storage cases may be designed as a docking and charging station that is kept at home.

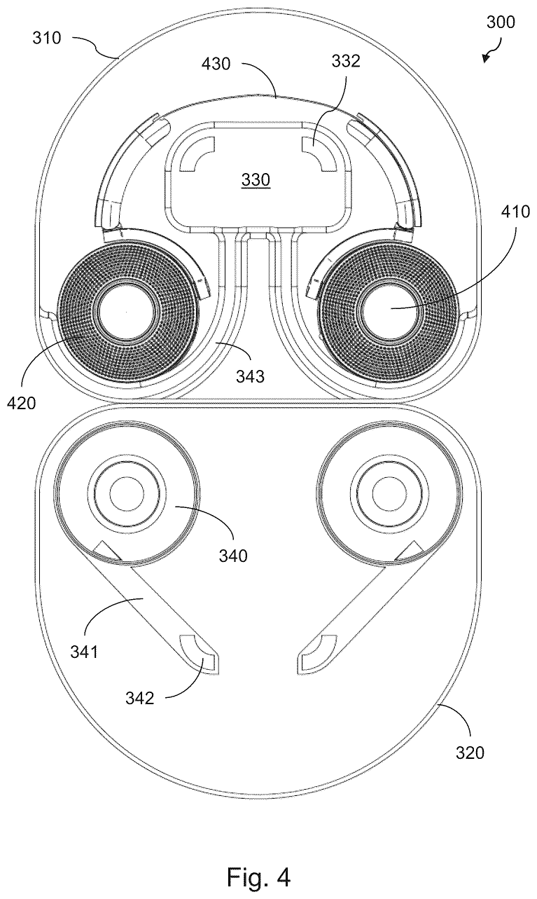

The portable air purifier 400 of FIGS. 3 and 4 is integrated in a pair of headphones. The two air inlets 420 of the air purifier 400 are positioned in the outer surfaces of two speaker assemblies 410. The air outlets of the air purifier 400 are not visible in these Figures but are provided in a side surface of the same speaker assemblies 410 and are configured to be connected to a visor (not shown) that, in use, may be positioned in front of the user's mouth. The storage case 300 comprises two docking bays 340 designed to receive the two respective speaker assemblies 410. Each docking bay 340 comprises a port 344 that is positioned to couple to the air outlet of the air purifier 400 when the air purifier 400 is in its docking position. This port 344 is connected to the sensor inlet of the test device 330 through a tract 343 that is formed in the bottom portion 310 of the storage case 300. In the cover 320 of the storage case 300, a similar tract 341 is provided with a port 342 that, when the cover 320 is closed, couples to the sensor outlet 332 of the test device 330. The docking bay 340 is designed in such a way that the air inlet 420 of the portable air purifier 400 is fully covered by the docking bay 340 when the cover 320 is closed. In this way, the closed loop through the air purifier 400 and the test device 330 that is needed for testing the filter condition is achieved by placing the air purifier 400 in the docking bay 340 and closing the storage case 300.

The storage case 300 may further comprise a power source that is part of or coupled to the test device 330 for providing electrical power thereto. This power source may be a rechargeable battery and/or a power connector for connecting to a power socket or external power source. If the storage case 300 comprises a rechargeable battery, this may be used for charging the portable air purifier 400 too. In addition to its protective and filter testing functionality, it may thus help to increase the amount of time the air purifier 400 can be used without having to connect it to a wall socket.

Preferably, the storage case 300 has some additional storage space for, in addition to the headphones/air purifier combination 400 holding useful accessories such as the visor of the air purifier, power or data cables for connection to a pc or an external power source, etc. According to a special embodiment of the invention, the storage case 300 further comprises at least one light source for emitting light in a violet portion of the visual spectrum, the at least one light source being arranged in such a way as to illuminate at least a portion of the portable air purifier. The emitted light preferably has a wavelength of about 405 nm, which is very suitable for decontaminating, e.g., the nozzles of the air purifier. Light guides inside the storage case may be configured to guide the light emitted by the at least one light source to those portions of the portable air purifier that are either likely to be contaminated or to pass on any contamination to the user.

The violet portion of the visual spectrum is typically defined as spanning the range of about 380 to 450 nm. The light used may thus, e.g., have a wavelength of about 405 nm. Light of these wavelengths is known to be very effective in killing any microbes that may have accumulated on the illuminated surfaces. The use of violet visible light for this particular implementation brings a number of advantages that are not found in UV or near UV light. For example, the low energy visible light does not damage the material of the surfaces it illuminates. This is especially advantageous because most domestic appliances are at least partially made of plastics that are easily damaged by UV light. Another important advantage of the violet visible light is that no direct line of sight between the light source and the surface or part to be cleaned is needed. Indirect irradiation of the violet visible light helps to get rid of the microbial contamination too.

It is to be noted that emitting light in a violet portion of the visual spectrum as part of a decontamination process means that the emitted light contains a significant portion of light in that part of the electromagnetic spectrum and that the intensity of that significant portion is sufficient to have a useful anti-microbial and decontaminating effect. The emitted light does not need to be exclusively in the violet portion of the visual spectrum. As long as there is a sufficient intensity of light in that portion of the spectrum, and preferably at or around the 405 nm wavelength, for achieving a decontaminating effect, light of other parts of the electromagnetic spectrum may be emitted too. Further it is noted that, as part of the decontamination process, the intensity of the emitted light may vary over time. Such variations may be gradual and continuous or in the form of a pattern of light pulses. If pulsed light is used, the frequency, duration and intensity of the pulses may either be constant or varying.

The invention has been described above in relation to a number of different embodiments. It is to be noted that test device according to the invention, although particularly useful in combination with portable and wearable air purifiers, is equally useful to and compatible with other types of air purifiers. Further, features used in and described with reference to specific embodiments are combinable with other embodiments. The scope of the invention is only limited by the following claims.

1. A test device for an air filter comprised in an air channel of a portable air purifier, the test device comprising a sensor inlet and a sensor outlet, the sensor inlet being connectable to the air channel of the portable air purifier, the test device further comprising a filter condition sensor, positioned between the sensor inlet and the sensor outlet, and configured to analyse a condition of the air filter.

2. The test device according to claim 1, wherein also the sensor outlet is connectable to the air channel of the air purifier.

3. The test device according to claim 1, wherein the test device comprises an internal pneumatic track connecting the sensor inlet to the sensor outlet, the filter condition sensor being positioned along the internal pneumatic track, the test device further comprising a first pneumatic track, connected to the sensor inlet and configured to be coupled to an air outlet of the portable air purifier.

4. The test device according to claim 3, further comprising a second pneumatic track, connected to the sensor outlet and configured to be coupled to an air inlet of the portable air purifier.

5. The test device according to claim 1, wherein the filter condition sensor comprises a pressure sensor.

6. The test device according to claim 1, wherein the filter condition sensor comprises a particulate matter sensor.

7. The test device according to claim 1, wherein the filter condition sensor comprises a gas sensor.

8. The test device according to claim 1, further comprising a power source, coupled to the filter condition sensor for providing electrical power thereto.

9. The test device according to claim 8, further comprising a power connector, connected to the power source and configured to be connected to a power input of the portable air purifier.

10. The test device according to claim 1, further comprising a test controller, operatively coupled to the filter condition sensor and operative to control a compressor to produce an airflow through the air channel and the test device.

11. The test device according to claim 10, wherein the test device comprises the compressor.

12. The test device according to claim 10, wherein the compressor is comprised in the portable air purifier.

13. The test device according to claim 4, further comprising at least one docking bay, connected to the second pneumatic track and shaped for holding at least a part of the portable air purifier in a predetermined position, such that the air inlet of the portable air purifier is fully covered by the docking bay.

14. A combination of a portable air purifier and the test device according to claim 1, an air outlet of the portable air purifier being coupled to the sensor inlet and an air inlet of the portable air purifier being coupled to the sensor outlet.

15. The combination according to claim 14, wherein the portable air purifier further comprises a purifier controller, operatively coupled to the filter condition sensor of the test device and operative to control a compressor to produce an airflow through the air channel and the test device.

16. The combination according to claim 15, wherein the test device comprises the compressor.

17. The combination according to claim 15, wherein the compressor is comprised in the portable air purifier.

18. A storage case for a portable air purifier comprising the test device according to claim 1.

19. The storage case according to claim 18, further comprising a power source, coupled to the test device for providing electrical power thereto.

20. The storage case according to claim 18, further comprising at least one light source for emitting light in a violet portion of the visual spectrum, the at least one light source being arranged in such a way as to illuminate at least a portion of the portable air purifier.

21. The storage case according to claim 20, wherein the at least one light source is configured for emitting light with a wavelength of about 405 nm.

22. The storage case according to claim 20, further comprising a light guide configured to guide the light emitted by the at least one light source to the at least one portion of the portable air purifier.
